(19) United States
(12) Patent Application Publication
(10) Pub. No.: US 2023/0033911 A1
(43) Pub. Date: Feb. 2, 2023

(54) BIOACTIVE COMPOSITIONS AND METHODS OF USE THEREOF

(71) Applicant: Ingenious Ingredients, LP, Lewisville, TX (US)

(72) Inventors: Ralf Jäger, Whitefish Bay, WI (US); Martin Purpura, Spring, TX (US); Shawn D. Wells, Frisco, TX (US); Kylin Liao, Plano, TX (US)

(21) Appl. No.: 17/875,207

(22) Filed: Jul. 27, 2022

Related U.S. Application Data
(60) Provisional application No. 63/203,644, filed on Jul. 27, 2021.
(60) Provisional application No. 63/226,057, filed on Jul. 27, 2021.

(51) Int. Cl. A23L 33/175; A23L 33/00

(52) U.S. Cl. CPC A23L 33/175; A23L 33/30

(57) ABSTRACT

The disclosed compositions, systems and methods relate to a dietary supplement for human consumption and comprises a combination of paraxanthine and tyrosine and/or taurine and optionally other compounds that modulate the effects of a combination of paraxanthine and tyrosine and/taurine. Further disclosed are methods of use of the foregoing compositions for improvement of at least one of endurance performance, mood, vigor, lipolysis, energy expenditure, exercise performance, and/or decreased appetite.

### CROSS-REFERENCE TO RELATED APPLICATION(S)

This application claims priority to U.S. Provisional Application No. 63/203,644 filed Jul. 27, 2021, and entitled “COMBINATION OF PARAXANTHINE AND TYROSINE-BASED BIOACTIVE COMPOSITION AND METHOD OF USE THEREOF,” and U.S.

Provisional Application No. 63/226,057, filed Jul. 27, 2021 and entitled “COMBINATION OF PARAXANTHINE AND TAURINE-BASED BIOACTIVE COMPOSITION AND METHOD OF USE THEREOF, each of which is hereby incorporated by reference in its entirety under 35 U.S.C. § 119(e).

### TECHNICAL FIELD

The disclosed technology relates generally to compositions, methods, for enhancing muscle and/or cognitive functions through administration of compositions containing combinations of paraxanthine, tyrosine, and/or taurine.

### BACKGROUND

Caffeine is a bitter, white crystalline purine, a methylxanthine alkaloid, and is chemically related to the adenine and guanine bases of deoxyribonucleic acid (DNA) and ribonucleic acid (RNA). It is found in the seeds, nuts, or leaves of several plants native to Africa, East Asia and South America, and helps to protect them against predator insects and to prevent germination of nearby seeds. The most well-known source of caffeine is the coffee bean, a misnomer for the seed of Coffea plants.

Caffeine concentrations in coffee beverages can be quite variable. A standard cup of coffee is often assumed to provide 100 mg of caffeine, but a recent analysis of 14 different specialty coffees purchased at coffee shops in the US found that the amount of caffeine in 8 oz (˜240 ml) of brewed coffee ranged from 72-130 mg (McCusker, R. R., Goldberger, B. A. and Cone, E. J. 2003. Caffeine content of specialty coffees. J. Anal. Toxicol., 27: 520-522.). Caffeine in espresso coffees ranged from 58-76 mg in a single shot. Interestingly, the caffeine content of the same type of coffee purchased from the same store on six separate days varied from 130 to 282 mg per 8-oz serving. Many individuals experience problems with sleep, anxiety, and/or jitteriness with caffeine, which may be exacerbated by an unexpectedly high dose.

Thus, there is a need in the art to identify alternative chemical compounds and mixtures thereof that may provide benefits. It is also desirable to provide chemical compounds and mixtures thereof that may be used to provide a variety of benefits, varying by concentration, thus requiring production of fewer materials.

### BRIEF SUMMARY

Disclosed here are compositions comprising paraxanthine and tyrosine and/or taurine and methods of use thereof. In certain aspects, the paraxanthine and tyrosine are present in a ratio from about 1:4 to about 1:30.

In certain embodiments, the disclosed compositions comprise a further active ingredient,

selected from a group consisting of: gallic acid, (+)-catechin (C), (−)-epicatechin (EC), (+)-gallocatechin (GC), (−)-epigallocatechin (EGC), (−)-catechin gallate (CG), (−)-gallocatechin gallate (GCG), (−)-epicatechin gallate (ECG) and (−)-epigallocatechin gallate (EGCG), glycerides, propylene glycol, lauroyl macrogol, lauroyl macrogol derivatives, cocrystallization products of bioperine, piperine, black pepper, bergamottin, dihydroxybergamottin (CYP3A4), flavonoids (naringin, hesperidin, nobiletin, tangeretin, quercetin), pterostilbene, fisetin, phytosomes, salicin, fish oil (omega-3 fatty acids and specialized, small lipid pro-resolving epoxide derivatives), oxylipins, tart cherry, hill oil, astaxanthin, proteolytic enzymes, glucosamine sulfate, chondroitin sulfate, MSM (methylsulfonylmethane), SAMe (Sadenosylmethionine), ASU (avocado-soybean unsapponifiable fraction), cetyl myristoleate, Dolichos falcate, triterpenoids, Acacia catechu, Andrographis paniculata, Scutalleria baicalensis, Agmatine sulfate, Stinging Nettle, Sea Buckthorn, Curcumin, Cissus Quadrilangularis, Boswellia Serrata, Wasabia japonica (wasabi extract for Tea Tree Oil), Emu Oil, Arnica, Mangifera indica L. (Anacardiaceae), Lagenaria breviflora, Zingiber officinale (ginger & gingerols/shogaols), hoodia gordonii, caffeine, yohimbine, methylsynephrine, synephrine, theobromine, flavenoids, tocopherols, theophylline, alphayohimbine, conjugated linoleic acid (CLA), octopamine, evodiamine, passion flower, red pepper, cayenne, raspberry ketone, guggul, green tea, guarana, kola nut, beta-Phenethylamines, Acacia rigidula, forskolin (Coleus forskohlli), theophylline, synephrine, yohimbine, rhodiola, ashwagandha, ginseng, Ginkgo biloba, siberian ginseng, astragalus, licorice, green tea, reishi, dehydroepiandrosterone (DHEA), pregnenolone, N-acetyl-tyrosine, glucuronolactone, Acetyl-L-carnitine, 5-hydroxytryptophan, tryptophan, Phenethylamines, Sceletium tortuosum (and Mesembrine alkaloids), Dendrobium sp., Acacia rigidula, PQQ (Pyroloquinoline quinone), Ubiquinone(01), Nicotinamide riboside, picamilon, Huperzine A (Chinese clubmoss or Huperzia serrata, L-dopa, Mucuna pruriens, and forskolin (Coleus forskohlli), 2-(dimethylamino)ethanol (DMAE), DMAE bitartrate, Ornithine, Citrulline, Pyruvate, Eleutherococcus senticosus, D-Ribose, whey protein, Trimethylglycine, Arginine, HMB (β-hydroxy β-methylbutyrate), milk protein, Schisandra chinensis, Leucine, Betalains, Leucic Acid, L-Carnitine, Sodium Bicarbonate, Arachidonic acid, Beta-Alanine, Brassinosteroids, Hemp Protein, Alanylglutamine, Rhaponticum carthamoides, Casein, Ecdysteroids, Creatine, Branched-Chain Amino Acids, Beetroot, Coffee, Nitrate, Panax ginseng, Clenbuterol, Alpha-GPC, Valine, Colostrum, Trichopus zeylanicus, Ashwagandha, Terminalia arjuna, Eggs, Ursolic Acid, Isoleucine, medium-chain triglycerides, Glutamine, zinc, vitamin D, maca, Schizandra, nicotinamide mononucleotide (NMN), exogenous ketones, Ergothioneine, berberine, dihydroberberine and combinations thereof.

In certain embodiments, the composition comprises a combination of paraxanthine and tyrosine congeners or combination of paraxanthine and tyrosine analogs. In exemplary implementations, the tyrosine congener or analog is N-acetyl-L-tyrosine, glycyl-L-tyrosine, N-Acetyl-L-tyrosine ethyl ester or N-acetyl-L-tyrosine methyl ester. In further implementations, tyrosine is present in polymeric form and wherein the polymeric form is Dityrosine (Tyr-Tyr), Trityrosine (Tyr-Tyr-Tyr), Tetratyrosine (Tyr-Tyr-Tyr-Tyr) or a peptide containing the forgoing. In yet further implementations, the tyrosine is present as Lysyltyrosine or Leucine-Tyrosine. In even further implementations, tyrosine is present in a dipeptide having the structure L-Tyr-X, wherein X is an amino acid.

Further disclosed herein is method for athletic performance or energy in subject by administering to the subject a composition comprising an effective amount of paraxanthine and tyrosine. In certain embodiments, administration of paraxanthine and taurine produce a synergistic increase in athletic performance or energy in the subject, relative to the administration of paraxanthine or taurine alone. In certain implementations, paraxanthine is provided in an amount of from about 25 mg to about 400 mg and wherein tyrosine is provided in an amount 100-150 mg/kg bodyweight of the subject. According to certain embodiments, the subject experiences increased endurance or increased strength.

In certain implementations, ratio of the amount of paraxanthine and tyrosine administered to the subject is from about 1:10 to about 1:30. In further implementations, ratio of the amount of paraxanthine and tyrosine administered to the subject is from about 1:10 to about 1:10

In further embodiments, the composition is substantially free of caffeine.

Further disclosed herein is a method of improving cognitive function in a subject comprising administering to the subject a composition comprising an effective amount of paraxanthine and tyrosine. In certain embodiments, improved cognitive function in the subject is measured by an increase in one or more of: attention, information acquisition, information processing, working memory, short-term memory, long-term memory, anterograde memory, retrograde memory, memory retrieval, discrimination learning, decision-making, inhibitory response control, attentional set-shifting, delayed reinforcement learning, reversal learning, the temporal integration of voluntary behavior, speed of processing, reasoning, problem solving and/or social cognition. In certain embodiments, administration of the composition to the subject enhances mood in the subject. In further embodiments, administration of paraxanthine and tyrosine produce a synergistic enhancement in cognitive function in the subject, relative to the administration of paraxanthine or tyrosine alone.

Further disclosed herein is a method of enhancing energy or mood in a subject comprising administering to the subject a composition comprising an effective amount of paraxanthine and taurine, wherein the amount for paraxanthine administered to the subject is from about 25 mg to about 800 mg and wherein the amount for taurine administered to the subject is from about 100 mg to about 6000 mg and wherein the administration of paraxanthine and taurine produce a synergistic enhancement in energy and/or mood in the subject, relative to the administration of paraxanthine or taurine alone.

While multiple embodiments are disclosed, still other embodiments of the disclosure will become apparent to those skilled in the art from the following detailed description, which shows and describes illustrative embodiments of the disclosed compositions, systems and methods. As will be realized, the disclosed compositions, systems and methods are capable of modifications in various obvious aspects, all without departing from the spirit and scope of the disclosure. Accordingly, the drawings and detailed description are to be regarded as illustrative in nature and not restrictive.

### BRIEF DESCRIPTION OF THE FIGURES

FIG. 1 shows exemplary data demonstrating the effect of certain disclosed compositions on forelimb strength in mice.

### DETAILED DESCRIPTION

Before explaining at least one embodiment of the invention in detail, it is to be understood that the invention is not limited in its application to the details of construction and to the arrangements of the components set forth in the following description or illustrated in the drawings. The invention is capable of other embodiments and of being practiced and carried out in various ways. Also, it is to be understood that the phraseology and terminology employed herein are for the purpose of description and should not be regarded as limiting.

Ranges can be expressed herein as from “about” one particular value, and/or to “about” another particular value. When such a range is expressed, a further aspect includes from the one particular value and/or to the other particular value. Similarly, when values are expressed as approximations, by use of the antecedent “about,” it will be understood that the particular value forms a further aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, the term “subject” refers to the target of administration, e.g., an animal. Thus, the subject of the herein disclosed methods can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. In one aspect, the subject is a mammal. A patient refers to a subject afflicted with a disease or disorder. As used herein, the term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. In various aspects, the term covers any treatment of a subject, including a mammal (e.g., a human), and includes: (i) preventing the disease from occurring in a subject that can be predisposed to the disease but has not yet been diagnosed as having it; (ii) inhibiting the disease, i.e., arresting its development; or (iii) relieving the disease, i.e., causing regression of the disease. In one aspect, the subject is a mammal such as a primate, and, in a further aspect, the subject is a human.

The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.).

As used herein, the terms “effective amount” and “amount effective” refer to an amount that is sufficient to achieve the desired result or to have an effect on an undesired condition. For example, a “therapeutically effective amount” refers to an amount that is sufficient to achieve the desired therapeutic result or to have an effect on undesired symptoms, but is generally insufficient to cause unacceptable adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors well known in the medical arts. For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. In further various aspects, a preparation can be administered in a “prophylactically effective amount”; that is, an amount effective for prevention of a disease or condition.

As used herein, the term “synergistic effect” or grammatical variations thereof means and includes a cooperative action encountered in a combination of two or more active compounds in which the combined activity of the two or more active compounds exceeds the sum of the activity of each active compound alone.

The term “synergistically effective amount,” as used herein, means and includes an amount of two or more active compounds that provides a synergistic effect defined above.

As used herein, the term “substantially” refers to the complete or nearly complete extent or degree of an action, characteristic, property, state, structure, item, or result. For example, an object that is “substantially” enclosed would mean that the object is either completely enclosed or nearly completely enclosed. The exact allowable degree of deviation from absolute completeness may in some cases depend on the specific context. However, generally speaking the nearness of completion will be so as to have the same overall result as if absolute and total completion were obtained. The use of “substantially” is equally applicable when used in a negative connotation to refer to the complete or near complete lack of an action, characteristic, property, state, structure, item, or result. For example, a composition that is “substantially free of particles would either completely lack particles, or so nearly completely lack particles that the effect would be the same as if it completely lacked particles. In other words, a composition that is “substantially free of an ingredient or element may still actually contain such item as long as there is no measurable effect thereof.

As used herein, “cognitive function” refers to any higher order intellectual brain process or brain state, respectively, involved in learning and/or memory including, but not limited to, attention, information acquisition, information processing, working memory, short-term memory, long-term memory, anterograde memory, retrograde memory, memory retrieval, discrimination learning, decision-making, inhibitory response control, attentional set-shifting, delayed reinforcement learning, reversal learning, the temporal integration of voluntary behavior, and expressing an interest in one's surroundings and self-care, speed of processing, reasoning and problem solving and social cognition.

### Compositions

Disclosed are compositions comprising a combination of paraxanthine and tyrosine and the related uses thereof. Further disclosed herein are compositions comprising a combination of paraxanthine and taurine and the related uses thereof. Paraxanthine may be produced synthetically or may be isolated from a natural source or through fermentation. Paraxanthine isolated from such sources may be purified to 95% or greater purity. Optionally, less purification may be used such that combination of paraxanthine for 50%, or even less, of the material. In some embodiments, it may be preferable to utilize paraxanthine isolated from a natural source which may include other congeners of paraxanthine typically found in paraxanthine sources.

In certain embodiments, the composition is formulated such that a dose contains paraxanthine ranging from about 1 to about 1000 mg (e.g., about 1 mg, about 5 mg, about 10 mg, about 15 mg, about 20 mg, about 25 mg, about 30 mg, about 35 mg, about 40 mg, about 45 mg, about 50 mg, about 75 mg, 100, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, or about 1000 mg, and the like, or any range or value therein).

The chemical formula for tyrosine is C₉H₁₁NO₃, and has a molecular weight of 181.19. Tyrosine is a dietary amino acid. It is also synthesized by the body from phenylalanine or phenylethylamine. In addition to its value as an energy substrate and in protein synthesis, it is a precursor to numerous biogenic amines and neurotransmitters. Tyrosine crosses the blood—brain barrier, and enters neurons, where it gets metabolized into catecholamine neurotransmitters. In certain embodiments, the source of the tyrosine is a natural source. In further embodiments, the source of tyrosine is synthetic. In yet further embodiments, the tyrosine is produced through fermentation.

According to further embodiments, tyrosine is present as an ester (e.g., L-tyrosine ethyl ester, L-Tyrosine methyl ester). In yet further embodiments, tyrosine is provided through a tyrosine derivative (e.g., N-acetyl-L-tyrosine and/or glycyl-L-tyrosine). In still further embodiments, the tyrosine derivative is present as an ester (e.g. N-Acetyl-L-tyrosine ethyl ester and N-acetyl-L-tyrosine methyl ester). In yet further embodiments, tyrosine is present in a polymeric form. Examples include, but are not limited to, dityrosine (Tyr-Tyr), trityrosine (Tyr-Tyr-Tyr), tetratyrosine (Tyr-Tyr-Tyr-Tyr) or peptides containing the forgoing.

According to further embodiments, tyrosine is present in a dipeptide having the structure L-Tyr-X, wherein X is an amino acid. In exemplary implementations, tyrosine is present in the form of Lysyltyrosine or Leucine-Tyrosine.

The chemical formula for taurine is C₂H₇NO₃S and has a molecular weight of 125.14. Taurine is naturally derived from cysteine. Mammalian taurine synthesis occurs in the pancreas via the cysteine sulfinic acid pathway. In this pathway, cysteine is first oxidized to its sulfinic acid, catalyzed by the enzyme cysteine dioxygenase. Cysteine sulfinic acid, in turn, is decarboxylated by sulfinoalanine decarboxylase to form hypotaurine. Hypotaurine is enzymatically oxidized to yield taurine by hypotaurine dehydrogenase. Synthetic taurine is obtained by the ammonolysis of isethionic acid (2-hydroxyethanesulfonic acid), which in turn is obtained from the reaction of ethylene oxide with aqueous sodium bisulfite. A direct approach involves the reaction of aziridine with sulfurous acid. Taurine is essential for cardiovascular function and development and function of skeletal muscle, the retina, and the central nervous system. In certain embodiments, the source of the taurine is a natural source. In further embodiments, the source of taurine is synthetic. In yet further embodiments, the taurine is produced through fermentation.

In certain embodiments, taurine is present in an amount ranging from about 500 to about 6000 mg (e.g., about 500 mg, about 1000 mg, about 1,500 mg, about 2,000 mg, about 2,500 mg, about 3,000 mg, about 3,500 mg, about 4,0000 mg, about 4,500 mg, about 5,000 mg, about 5,500 mg, or about 6000 mg and the like, or any range or value therein).

In certain embodiments, the combination of paraxanthine and tyrosine and/or taurine may be combined with one or more other chemical compounds (e.g. other active ingredients), to provide a plurality of positive effects in a subject. By altering the dosage of the combination of paraxanthine and tyrosine and/or chemical compounds it is combined with, various physiological effects may be selected for. The compositions may provide primarily a single benefit, or may provide multiple benefits simultaneously. In certain embodiments, the combination of paraxanthine and tyrosine is combined with one or more additional active ingredients selected from: a group consisting of: gallic acid, (+)-catechin (C), (−)-epicatechin (EC), (+)-gallocatechin (GC), (−)-epigallocatechin (EGC), (−)-catechin gallate (CG), (−)-gallocatechin gallate (GCG), (−)-epicatechin gallate (ECG) and (−)-epigallocatechin gallate (EGCG), glycerides, propylene glycol, lauroyl macrogol, lauroyl macrogol derivatives, cocrystallization products of bioperine, piperine, black pepper, bergamottin, dihydroxybergamottin (CYP3A4), flavonoids (naringin, hesperidin, nobiletin, tangeretin, quercetin), pterostilbene, fisetin, phytosomes, salicin, fish oil (omega-3 fatty acids and specialized, small lipid pro-resolving epoxide derivatives), oxylipins, tart cherry, hill oil, astaxanthin, proteolytic enzymes, glucosamine sulfate, chondroitin sulfate, MSM (methylsulfonylmethane), SAMe (S adenosylmethionine), ASU (avocado-soybean unsapponifiable fraction), cetyl myristoleate, Dolichos falcate, triterpenoids, acacia catechu, Andrographis paniculata, Scutalleria baicalensis, Agmatine sulfate, Stinging Nettle, Sea Buckthorn, Curcumin, Cissus Quadrilangularis, Boswellia Serrata, Wasabia japonica (wasabi extract for Tea Tree Oil), Emu Oil, Arnica, Mangifera indica L. (Anacardiaceae), Lagenaria breviflora, Zingiber officinale (ginger & gingerols/shogaols), hoodia gordonii, caffeine, yohimbine, methylsynephrine, synephrine, theobromine, tocopherols, theophylline, alphayohimbine, conjugated linoleic acid (CLA), octopamine, evodiamine, passion flower, red pepper, cayenne, raspberry ketone, guggul, green tea, guarana, kola nut, beta-Phenethylamines, Acacia rigidula, forskolin (Coleus forskohlli), theophylline, synephrine, yohimbine, rhodiola, ashwagandha, ginseng, Ginkgo biloba, siberian ginseng, astragalus, licorice, green tea, reishi, dehydroepiandrosterone (DHEA), pregnenolone, N-acetyl-tyrosine, glucuronolactone, Acetyl-L-carnitine, 5-hydroxytryptophan, tryptophan, Phenethylamines, Sceletium tortuosum (and Mesembrine alkaloids), Dendrobium sp., Acacia rigidula, PQQ (Pyroloquinoline quinone), Ubiquinone(01), Nicotinamide riboside, picamilon, Huperzine A (Chinese clubmoss or Huperzia serrata, L-dopa, Mucuna pruriens, forskolin (Coleus forskohlli), 2-(dimethylamino)ethanol (DMAE), DMAE bitartrate, medium chain triglycerides, creatine, citrulline, arginine, lions mane, cordyceps, leucine, isoleucine, valine, BAIBA, ergothioneine, grains of paradise, Kanna, Huperzine A, ketones, Maca, ginseng, ashwagandha, rhodiola, theanine and combinations thereof.

In certain embodiments, paraxanthine and tyrosine are present in about equal amounts. In these embodiments, paraxanthine and tyrosine each comprise about 50% of the combined weight of paraxanthine and tyrosine within the composition, on a w/v basis. In certain further embodiments, the range may be from at least 10% of paraxanthine to 90% and 90% of tyrosine to 10%, respectively.

In further embodiments, paraxanthine and tyrosine are present at a ratio of from 1:4 to about 1:30. In still further embodiments, paraxanthine and tyrosine are present at a ratio from about 1:4 to about 1:10.

In certain embodiments, tyrosine is administered to the subject at dose ranging from about 100-150 mg/kg bodyweight of the subject.

In certain embodiments, paraxanthine and taurine are present in about equal amounts. In these embodiments, paraxanthine and taurine each comprise about 50% of the combined weight of paraxanthine and taurine within the composition, on a w/v basis. In certain further embodiments, the range may be from at least 10% of paraxanthine to 90% and 90% of taurine to 10%, respectively. In further embodiments, taurine and paraxanthine are present at a ratio of about 4:1 to about 1:4.

In certain embodiments, the composition is formulated such that a dose contains paraxanthine ranging from about 1 to about 1000 mg (e.g., about 1 mg, about 5 mg, about 10 mg, about 15 mg, about 20 mg, about 25 mg, about 30 mg, about 35 mg, about 40 mg, about 45 mg, about 50 mg, about 75 mg, 100, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, or about 1000 mg, and the like, or any range or value therein).

In certain embodiments, the composition is formulated such that a dose contains tyrosine ranging from about 500 to about 13,500 mg (e.g., about 500 mg, about 1000 mg, about 1,500 mg, about 2,000 mg, about 2,500 mg, about 3,000 mg, about 3,500 mg, about 4,0000 mg, about 4,500 mg, about 5,000 mg, about 7,500 mg, 10,000, about 13,500 mg, and the like, or any range or value therein).

Depending upon the subject to be treated and the route of administration, the compounds of the invention may be administered at varying doses. Although doses will vary from subject to subject, suitable daily doses are in the range of about 1 to about 1000 mg (e.g., about 1 mg, about 5 mg, about 10 mg, about 15 mg, about 20 mg, about 25 mg, about 30 mg, about 35 mg, about 40 mg, about 45 mg, about 50 mg, about 75 mg, 100, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, or about 1000 mg, and the like, or any range or value therein) per subject, administered in single or multiple doses.

In certain embodiments, the composition is formulated such that a dose contains paraxanthine each ranging from about 1 to about 1000 mg (e.g., about 1 mg, about 5 mg, about 10 mg, about 15 mg, about 20 mg, about 25 mg, about 30 mg, about 35 mg, about 40 mg, about 45 mg, about 50 mg, about 75 mg, 100, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, or about 1000 mg, and the like, or any range or value therein) and taurine ranging from 400 to about 3000 mg (e.g., about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1500 mg, about 2000 mg, about 2500 mg, or about 3000 mg and the like, or any range or value therein).

### Nutritional Supplements

The compositions of the disclosure may take the form of dietary supplements or may themselves be used in combination with dietary supplements, also referred to herein as food supplements.

Nutritional supplements may be found in many forms such as tablets, capsules, soft gels, gel caps, liquids, or powders. Some dietary supplements can help ensure an adequate dietary intake of essential nutrients; others may help reduce risk of disease.

### Food Products

The compositions of the disclosure may take the form of a food product. Here, the term “food” is used in a broad sense and covers food and drink for humans as well as food and drink for animals (i.e. a feed). Preferably, the food product is suitable for, and designed for, human consumption.

The food may be in the form of a liquid, solid or suspension, depending on the use and/or the mode of application and/or the mode of administration.

When in the form of a food product, the composition may comprise or be used in conjunction with one or more of: a nutritionally acceptable carrier, a nutritionally acceptable diluent, a nutritionally acceptable excipient, a nutritionally acceptable adjuvant, a nutritionally active ingredient.

By way of example, the compositions of the disclosure may take the form of one of the following: A fruit juice; a beverage comprising whey protein: a health or herbal tea, a cocoa drink, a coffee drink, a yoghurt and/or a drinking yoghurt, a cheese, an ice cream, a desserts, a confectionery, a biscuit, a cake, cake mix or cake filling, a snack food, a fruit filling, a cake or doughnut icing, an instant bakery filling cream, a filling for cookies, a ready-to-use bakery filling, a reduced calorie filling, an adult nutritional beverage, an acidified soy/juice beverage, a nutritional or health bar, a beverage powder, an energy drink, a sublingual, a gummy, a calcium fortified soy milk, or a calcium fortified coffee beverage.

### Food Ingredients

Compositions of the present disclosure may take the form of a food ingredient and/or feed ingredient.

As used herein the term “food ingredient” or “feed ingredient” includes a composition which is or can be added to functional foods or foodstuffs as a nutritional and/or health supplement for humans and animals.

The food ingredient may be in the form of a liquid, suspension or solid, depending on the use and/or the mode of application and/or the mode of administration.

### Functional Foods

Compositions of the disclosure may take the form of functional foods. As used herein, the term “functional food” means food which is capable of providing not only a nutritional effect but is also capable of delivering a further beneficial effect to the consumer.

Accordingly, functional foods are ordinary foods that have components or ingredients (such as those described herein) incorporated into them that impart to the food a specific function—e.g. medical or physiological benefit—other than a purely nutritional effect.

Although there is no legal definition of a functional food, most of the parties with an interest in this area agree that they are foods marketed as having specific health effects beyond basic nutritional effects.

Some functional foods are nutraceuticals. Here, the term “nutraceutical” means a food which is capable of providing not only a nutritional effect and/or a taste satisfaction, but is also capable of delivering a therapeutic (or other beneficial) effect to the consumer. Nutraceuticals cross the traditional dividing lines between foods and medicine.

### Medical Foods

Compositions of the present disclosure may take the form of medical foods. By “medical food” it is meant a food which is formulated to be consumed or administered with or without the supervision of a physician and which is intended for a specific dietary management or condition for which distinctive nutritional requirements, based on recognized scientific principles, are established by medical evaluation.

### Methods of Use

In certain embodiments, paraxanthine may be combined with tyrosine and/or taurine, and in certain embodiments, one or more other chemical compounds (e.g. other active ingredients), to provide a plurality of positive effects in a subject. By altering the dosage of paraxanthine and/or chemical compounds it is combined with, various physiological effects may be selected for. The compositions may provide primarily a single benefit or may provide multiple benefits simultaneously. Depending upon the subject to be treated and the route of administration, the compounds of the invention may be administered at varying doses. Although doses will vary from subject to subject, suitable daily doses are in the range of about 1 to about 14,500 mg (e.g., about 1 mg, about 5 mg, about 10 mg, about 15 mg, about 20 mg, about 25 mg, about 30 mg, about 35 mg, about 40 mg, about 45 mg, about 50 mg, about 75 mg, 100, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1,500 mg, about 2,000 mg, about 2,500 mg, about 3,000 mg, about 3,500 mg, about 4,0000 mg, about 4,500 mg, about 5,000 mg, about 7,500 mg, 10,000, about 13,500 mg, about 14,000, or about 14,5000 mg and the like, or any range or value therein) per subject, administered in single or multiple doses.

In certain embodiments, paraxanthine and tyrosine and/or taurine may be administered to the subject as part of a single composition. In further embodiments, paraxanthine and tyrosine and/or taurine as separate compositions administered simultaneously or sequentially.

Advantageously, compositions of the present disclosure may be administered in single doses, e.g. once daily or more seldom, or in a total daily dosage administered in divided doses of two, three or four times daily. In certain embodiments, the composition is administered as needed (e.g., when the subject is in need of enhance energy, athletic or cognitive performance or the like).

### Athletic Performance

Further disclosed herein is a method for enhancing performance or energy in subject, comprising administering to the subject a composition disclosed herein. As used herein the term “enhancing performance” is intended to mean any improvement in performance. Performance can be assessed in any manner. Certain enhancements are readily measured. For example, in a timed-event, an improved time can assess an enhanced performance. Certain performance enhancing properties can be judged subjectively by the athlete or performer or an observer. In these instances, an enhanced performance means that the performance was perceived subjectively to be improved, magnified, faster, better and the like. In certain embodiments, the disclosed methods are used to enhance athletic performance. “Athletic performance” refers to any professional or recreational activity wherein the performer, for example an athlete, exerts a physical act, such as running, swimming, golf, bowling, archery, football, baseball, basketball, soccer, hiking, cycling, dancing and the like. In certain athletic performance is improved through in improvement of endurance in the subject. In other words, administration of the disclosed compositions improves a subject's level of endurance, thereby enhancing the subject's athletic performance. In further embodiments, administration of the composition to the subject increases cognitive performance which thereby improves athletic performance.

In certain embodiments, upon administration of the composition, the subject experiences improvement of at least one of mood, energy, focus, concentration or sexual desire or a reduction of at least one of anxiety, fatigue, perception of effort or perception of pain.

In further embodiments, upon continued administration to the subject, the composition does not create dependence in the subject and/or withdrawal effect in the subject when continued use is ceased.

Further disclosed herein is a method of increasing athletic endurance in a subject comprising administering to the subject a composition disclosed herein. In certain implementations, the composition administered to the subject comprises paraxanthine and tyrosine. In exemplary implementations, the administration of paraxanthine and tyrosine produce a synergistic increase athletic endurance in the subject, relative to the administration of paraxanthine or tyrosine alone.

Further disclosed herein is a method of increasing athletic endurance in a subject comprising administering to the subject a composition disclosed herein. In certain implementations, the composition administered to the subject comprises paraxanthine and tyrosine. In exemplary implementations, the administration of paraxanthine and taurine produce a synergistic increase athletic endurance in the subject, relative to the administration of paraxanthine or taurine alone.

According to further embodiments, administration of the disclosed composition to the subject increases the subject's perceived level of energy. In exemplary implementations, the subject experiences an increase in energy of at least about 5 percent. According to certain embodiments, the composition administered further comprises (in addition to paraxantine and/or tyrosine, and/or taurine) at least one ingredient selected from the group consisting of L-theanine, phosphatidylcholine, alpha-GPC (L-alpha glycerylphosphorylcholine), Citicoline (Cytidine diphosphate choline (CPD Choline)), Choline Bitartrate, Bacopa Monnieri, Phosphatidylserine, pilocarpine, and cevimeline Amburana cearensis, Lippia sidoides, Paullinia cupana, Plathymiscium floribundum, tetrahydrocurcumin, and Solanum asperum and/or combinations thereof, caffeine, theobromine, naringin, hesperidin, 2-(dimethylamino)ethanol (DMAE), DMAE bitartrate, huperzine A, theacrine, methylliberine, B12, sulbutiamine, magnolia bark, ketones, MCTs, omega 3's, lutein, zeaxanthin, and n-acetyl-tyrosine, acetyl-1-carnitine and/or combinations thereof.

In certain embodiments, the subject's perceived level of energy is increased by between about 2% and about 50%. In further embodiments, the subject's perceived level of energy is increased by between about 5% and about 30%. In yet further embodiments, the subject's perceived level of energy is increased by between about 10% and about 25%.

### Muscle Function

Further disclosed herein is a method for increasing muscle function in a subject by administering to the subject a composition disclosed herein. In certain aspects, disclosed herein are methods to promote muscle growth through the administration of an effective amount of one or more compositions disclosed herein. In certain further aspects, administration of effective amounts of the disclosed compositions results in greater level of muscle protein synthesis (MPS) in the subject. In still further aspects, administration of effective amounts of the disclosed compositions results in improved muscle accretion in the subject.

In certain aspects, disclosed herein are methods to promote muscle growth through the administration of an effective amount of one or more compositions disclosed herein. In certain further aspects, administration of effective amounts of the disclosed compositions results in greater level of muscle protein synthesis (MPS) in the subject. In still further aspects, administration of effective amounts of the disclosed compositions results in improved muscle accretion in the subject.

According to certain embodiments, compositions disclosed herein may be administered in conjunction with a strength training regime. As will be appreciated by a person having skill in the art, administration of effective amounts of the disclosed compositions results in improved strength and improved athletic performance/ergogenesis in the subject.

In one aspect, the disclosed compounds inhibit muscle atrophy. In a further aspect, the disclosed compounds increase muscle mass. In a still further aspect, the disclosed compounds induce muscle hypertrophy. In a yet further aspect, the disclosed compounds inhibit of muscle atrophy and increase muscle mass. In an even further aspect, the disclosed compounds inhibit of muscle atrophy and induce muscle hypertrophy. In a further aspect, the inhibition of muscle atrophy is in a subject. In an even further aspect, the increase in muscle mass is in a subject. In a still further aspect, the subject is a mammal. In a yet further aspect, the mammal is a human.

In certain aspects, administration of the disclosed compositions is effective at preventing or treating age-related muscle atrophy or sarcopenia. In further aspects, administration of the disclosed compositions is effective at preventing or treating muscle atrophy associated with muscle immobilization, such as that which frequently occurs with casting of fractured bones. In further aspects, administration of the disclosed compositions is effective at preventing or treating muscle atrophy associated with disease, such as cancer, also known as cachexia.

According to certain aspects the composition is administered to a subject that has sarcopenia. In various aspects, the composition is administered in a therapeutically effective amount. In further aspects, the composition is administered at prophylactically effective amount, (e.g. to a subject at risk for developing sarcopenia, cachexia, or immobilization induced atrophy).

In certain aspects, the composition further comprises one or more additional active ingredient to further enhance muscle strength, size, and/or muscle function. In certain embodiments, the one or more additional active ingredient is an amino acid. According to certain embodiments, the amino acid is selected from a group of branched-chain amino acids (BCAA), including, but not limited to, isoleucine, leucine, and valine. In further embodiments, the amino acid is selected from the group of essential amino acids, including, but not limited to, histidine, isoleucine, leucine, lysine, methionine, phenylalanine, threonine, tryptophan, and valine. In still further embodiments, the amino acid is selected from the group of conditionally essential amino acids including, but not limited to, arginine, cysteine, glutamine, glycine, proline, ergothioneine, and tyrosine. According to the certain embodiments, the conditionally essential amino acid is tyrosine. In still further embodiments, the amino acid is selected from the group of non-essential amino acids including, but not limited to, alanine, aspartic acid, asparagine, glutamic acid, serine, selenocysteine and pyrrolysine. In yet further embodiments, the amino acid derivative is selected from the group of creatine, carnitine, beta-alanine, taurine, beta-hydroxy beta-methylbutyrate L-Arginine, omega-3 fatty acids, Vitamin D, whey protein, BAIBA, and other protein extracts from animal, plant or fermentation sources.

According to exemplary aspects of these embodiments, that may reduce fatigue, improve energy, increase mobility, and improve alertness. In further embodiments, administration of the disclose compositions is cardio protective. In further embodiments, administration of the disclose compositions improves muscle contractions and muscle performance. In exemplary aspects, of these embodiments, muscle performance is enhanced through increasing potassium (K+) transport into skeletal muscle. In further aspects, muscle performance is enhanced through increasing intracellular calcium (e.g., via ryanodine receptor (RyR) activation).

In certain aspects of the foregoing embodiments wherein the composition comprises effective amounts of tyrosine and paraxanthine, the administration of paraxanthine and tyrosine produce a synergistic increase in muscle size and/or function in the subject, relative to the administration of paraxanthine or tyrosine alone.

In certain aspects of the foregoing embodiments wherein the composition comprises effective amounts of taurine and paraxanthine, the administration of paraxanthine and taurine produce a synergistic increase in muscle size and/or function in the subject, relative to the administration of paraxanthine or taurine alone.

### Cognitive Function

Disclosed herein is a method of enhancing cognitive function in a subject comprising administering to the subject a composition disclosed herein. In certain embodiments, improved cognitive function is measured by an increase in one or more of: attention, information acquisition, information processing, working memory, short-term memory, long-term memory, anterograde memory, retrograde memory, memory retrieval, discrimination learning, decision-making, inhibitory response control, attentional set-shifting, delayed reinforcement learning, reversal learning, the temporal integration of voluntary behavior, speed of processing, reasoning, problem solving and/or social cognition.

In certain embodiments, administration of the disclosed composition increases working memory.

In further embodiments, administration of the disclosed composition increases attention.

According to certain embodiments, composition of the instantly disclosed methods to enhance cognitive function further comprise, N-acetyl-tyrosine, taurine, huperzine A, acetyl-1-carnitine, CDP choline, Alpha GPC, choline bitrate, choline citrate, B12, caffeine, methyllliberine, theacrine, paraxanthine, theobromine, ashwagandha, rhodiola, lutein, zeaxanthin, fish oil, creatine, ginseng, lions mane, niacin, cordyceps, theanine, B-vitamins, GABA, sulbutiamine, vinpocetine, adenosine triphosphate, inositol, enhanced arginine silicate, nitrates, electrolytes, hesperidin and derivatives of hesperidin and/or bacopa.

In certain embodiments, the subject has experience age-related cognitive decline. In exemplary implementations, administration of the composition to the subject increases the level BDNF in the subject. According to certain embodiments, administration of the composition to the subject increases brain derived neurotrophic factor (BDNF) levels in the subject. In exemplary implementations, BDNF levels are increased by from about 5% to about 40%. In further embodiments, BDNF levels are increased by at least about 15%. In further embodiments, administration of the composition to the subject increases other neurotrophic factors such as neuronal growth factor (NGF). In still further embodiments, administration of the composition to the subject increases levels of mTOR in the CNS.

### Methods of Treatment

Further disclosed herein is a method of treating a condition in a subject in need thereof by administering to the subject a composition disclosed herein. In certain embodiments, the condition is selected from narcolepsy, epilepsy, attention deficit disorders, attention deficit hyperactivity syndrome (ADHD), cognitive deficit disorders, palsies, uncontrolled anger, migraine, substance abuse addictions, eating disorders, depression, anxiety disorders, traumatic head injury (TBI), Parkinson's disease, Alzheimer's, and dementia.

Further disclosed herein is a method for treating a mood disorder by administering to a subject in need thereof a composition disclosed herein. In certain embodiments, the mood disorder is selected from clinical depression, postnatal depression or postpartum depression, perinatal depression, atypical depression, melancholic depression, psychotic major depression, catatonic depression, seasonal affective disorder, dysthymia, double depression, depressive personality disorder, recurrent brief depression, minor depressive disorder, bipolar disorder or manic depressive disorder, depression caused by chronic medical conditions, comorbid depression, treatment-resistant depression, refractory depression, suicidality, suicidal ideation, or suicidal behavior. In some embodiments, the method described herein provides therapeutic effect to a subject suffering from depression (e.g., moderate or severe depression). In some embodiments, the mood disorder is associated with a disease or disorder described herein.

In certain embodiments, the mood disorder is depression. In exemplary implementations, subject has been diagnosed with depression or is at risk of depression.

Further disclosed herein is a method for treating an anxiety disorder in a subject in need thereof by administering to a subject in need thereof a composition disclosed herein. In certain embodiments, the anxiety disorder is selected from: generalized anxiety disorder, panic disorder, obsessive compulsive disorder, phobia, post-traumatic stress disorder). As will be appreciated by those skilled in the art, anxiety disorder is a blanket term covering several different forms of abnormal and pathological fear and anxiety.

According to certain embodiments, the composition is administered in a therapeutically effective amount. In further embodiments, the composition is administered in a prophylactically effective amount.

In certain embodiments, the composition used in the method of treating a mood disorder or anxiety disorder further comprises at least one ingredient selected from the group consisting of L-theanine, phosphatidylcholine, alpha-GPC (L-alpha glycerylphosphorylcholine), Citicoline (Cytidine diphosphate choline (CPD Choline)), Choline Bitartrate, Bacopa Monnieri, Phosphatidylserine, pilocarpine, and cevimeline Amburana cearensis, Lippia sidoides, Paullinia cupana, Plathymiscium floribundum, tetrahydrocurcumin, and Solanum asperum and/or combinations thereof, caffeine, theobromine, naringin, hesperidin, 2-(dimethylamino)ethanol (DMAE), DMAE bitartrate, magnolia bark, theanine, phosphatidylserine, ashwagandha, rhodiola, macuna, sceletium tortuosa, 5-HTP, tryptophan, saffron, Vitamin D, SAMe, lions mane and/or huperzine A.

Further disclosed herein is a method for treating or preventing age-related cognitive decline in a subject in need thereof, comprising administering to the subject an effective amount of a composition disclosed herein. In certain embodiments, administration of the composition increases one or more of attention, information acquisition, information processing, working memory, short-term memory, long-term memory, anterograde memory, retrograde memory, memory retrieval, discrimination learning, decision-making, inhibitory response control, attentional set-shifting, delayed reinforcement learning, reversal learning, the temporal integration of voluntary behavior, speed of processing, reasoning, problem solving and/or social cognition..

According to certain embodiments, the composition disclosed herein are used in the treatment of one or more medical conditions in a subject in need thereof. In certain implementations, the disclosed composition is administered to a subject suffering from narcolepsy, sleep apnea, and shift work sleep disorder, insomnia epilepsy, attention deficit disorders, attention deficit hyperactivity syndrome (ADHD), cognitive deficit disorders, palsies, uncontrolled anger, migraine, substance abuse addictions, eating disorders, depression, anxiety disorders, traumatic head injury (TBI), Parkinson's disease, Alzheimer's, and/or dementia.

In certain aspects, the disclosed compositions are a neuroprotective agent. In certain embodiments, administration of the disclosed compositions to a subject in need thereof is neuroprotective. In exemplary aspects of these embodiments, this neuroprotection is in the form of protecting against dopaminergic cell death.

According to further embodiments, disclosed compositions are useful for the treatment of geriatric depression. In exemplary embodiments, the compositions are effective in treating subjects suffering from geriatric depression an essential, vascular or traumatic origin. And of the mental decay in the elderly.

The administration of the disclosed compositions to a subject may include any method of providing a pharmaceutical preparation to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, sublingual administration, intradermal administration, buccal administration, and parenteral administration, including injectable such as intravenous administration, intra-arterial administration, intramuscular administration, and subcutaneous administration. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

In another embodiment, a combination of paraxanthine and tyrosine and/or taurine may be used at lower dosage levels and/or in conjunction with compounds that modulate or antagonize its activity. Such compositions may induce an improved endurance performance, mood, vigor, lipolysis, energy expenditure, exercise performance, and/or decreased appetite.

An advantage of using the disclosed compositions is the reduced likelihood that a person develops a tolerance to chemical compositions. That is, a person may not become desensitized to the effects induced. According to certain aspects, the disclosed combination of paraxanthine and tyrosine and/or taurine containing compositions has at least the following distinct advantages over the administration of compositions containing comparable doses of caffeine. The combination of paraxanthine and tyrosine and/or taurine has substantially lower toxicity. The combination of paraxanthine and tyrosine has greater stability (e.g. does not lose potency over time to the same extent as caffeine). A composition containing a combination of paraxanthine and tyrosine and/or taurine is a more potent wake-promoting agent (in certain embodiments, via adenosine receptor antagonism). Further, compositions containing a combination of paraxanthine and tyrosine and/or taurine enhance striatal dopaminergic tone. Still further, the combination of paraxanthine and tyrosine and/or taurine does not produce sleep rebound. Further, a combination of paraxanthine and tyrosine and/or taurine does not produce withdrawal effects upon cessation of use, as frequently occurs with caffeine. Yet further, a combination of paraxanthine and tyrosine and/or taurine does not enhance anxiety. Still further, a combination of paraxanthine and tyrosine and/or taurine is less bitter than caffeine. Even further, a combination of paraxanthine and tyrosine and/or taurine is effective for a larger portion of the population than caffeine. In another embodiment, a combination of paraxanthine and tyrosine and/or taurine may be used at higher dosage levels and/or with synergistic compounds.

These compositions may increase a person's basal/resting metabolic rate, increase thermogenesis, decrease appetite, enhance cognitive performance, increase alpha wave brain activity, and/or induce euphoria. Without being bound by theory, the inventors believe that at higher dosage levels, a composition containing a combination of paraxanthine and tyrosine and/or taurine may be noradrenergic and dopaminergic and may exhibit increased adenosine receptor inhibition.

In another embodiment, paraxanthine and tyrosine and/or taurine are combined with ephedrine, caffeine, salicylic acid or the like. The foregoing combinations may produce a synergistic effect with the stimulating effects of combination of paraxanthine and tyrosine. For example, in certain embodiments, paraxanthine and tyrosine may be combined with much lesser amounts of caffeine in order to modulate the excessive stimulatory effects of caffeine, thereby stabilizing heart rate and other metabolic activity. That is, a combination of paraxanthine and tyrosine and/or taurine and caffeine may result in a composition that imparts the increased focus and energy induced by caffeine, but without the higher heart rate and blood pressure due to modulation of caffeine's effects by combination of paraxanthine and tyrosine and/or taurine. Thus, the combination may result in heightened awareness and calmness without the jitters caffeine may cause.

In another embodiment, combination of paraxanthine and tyrosine may be used as a topical agent for incorporation into body creams or lotions to produce a cream or lotion for lightening skin, firming skin, and/or improving skin elasticity. A topical agent containing a combination of paraxanthine and tyrosine and/or taurine may also be used to promote localized transdermal fat loss. Such a composition may also be used in a cream or lotion to promote localized enhanced metabolism and/or enhanced thermogenesis.

According to further embodiments, paraxanthine and tyrosine may be combined with one or more of analgesics and/or anti-inflammatory agents. In exemplary implementations, paraxanthine and tyrosine and/or taurine are combined with ibuprofen, salicylic acid, anti-inflammatory agents, salicin, fish oil (omega-3 fatty acids and specialized, small lipid pro-resolving derivatives), tart cherry, krill oil, astaxanthin, proteolytic enzymes, glucosamine sulfate, chondroitin sulfate, MSM (methylsulfonylmethane), SAMe (S-adenosylmethionine), ASU (avocado-soybean unsapponifiable fraction), cetyl myristoleate, Dolichos falcate and/or triterpenoids.

The dosage of combination of paraxanthine and tyrosine and/or taurine may range from about 100 mg to about 3000 mg. In another embodiment, the range may be from about 500 mg to about 2500 mg. In further embodiments, the combined dose of paraxanthine and tyrosine is about 600 mg. In another embodiment, the range may be from at least 10% of paraxanthine to 90% and 90% of taurine to 10%, respectively.

In certain embodiments, the composition comprises paraxanthine and tyrosine at a ratio of about 1:5. In certain embodiments, the amount of paraxanthine provided is about 2 mg to about 800 mg and the amount of tyrosine provided is about 500 mg to about 2000 mg.

In exemplary implementations, the composition is administered at a dose of about 100 mg paraxanthine and about 500 mg tyrosine.

In another embodiment, a combination of paraxanthine and tyrosine and/or taurine is combined with one or more bioavailability enhancers. In exemplary embodiments, bioavailability enhancers include, but are not limited to: bioperine, piperine, black pepper, bergamottin, dihydroxybergamottin (CYP3A4 inhibitors), flavonoids (including hesperidin, naringin, tangeritin, quercetin and nobiletin both in isolation and in combination), pterostilbenes, fisetin, nanoencapsulation, microencapsulation, liposomes and/or phytosomes. The enhancers that are combined with combination of paraxanthine and tyrosine may depend on which qualities of combination of paraxanthine and tyrosine and/or taurine are desired for a particular use.

In another embodiment, the combination of paraxanthine and tyrosine may be administered using one or more delivery methods, including, for example transdermal patches and/or creams, ready to mix powders, intravenous methods, capsules, tablets, liquid (including liquids for mixing with other beverages), softgels, shot format, and/or cosmetic applications including soaps, lotions and shampoos, combination of paraxanthine and tyrosine's anti-inflammatory qualities may be desired for a variety of topical applications.

The administration of the disclosed compositions to a subject may include any method of providing a pharmaceutical preparation to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, sublingual administration, intradermal administration, buccal administration, and parenteral administration, including injectable such as intravenous administration, intra-arterial administration, intramuscular administration, and subcutaneous administration. Administration can be continuous or intermittent. In various aspects, a preparation can be administered therapeutically; that is, administered to treat an existing disease or condition.

In further various aspects, a preparation can be administered prophylactically; that is, administered for prevention of a disease or condition.

Various aspects and embodiments of the present invention are defined by the following numbered clauses:

1. A composition comprising paraxanthine and tyrosine.
2. The composition of clause 1, further comprising one or more active ingredient, selected from a group consisting of: gallic acid, (+)-catechin (C), (−)-epicatechin (EC), (+)-gallocatechin (GC), (−)-epigallocatechin (EGC), (−)-catechin gallate (CG), (−)-gallocatechin gallate (GCG), (−)-epicatechin gallate (ECG) and (−)-epigallocatechin gallate (EGCG), glycerides, propylene glycol, lauroyl macrogol, lauroyl macrogol derivatives, cocrystallization products of bioperine, piperine, black pepper, bergamottin, dihydroxybergamottin (CYP3A4), flavonoids (naringin, hesperidin, nobiletin, tangeretin, quercetin), pterostilbene, fisetin, phytosomes, salicin, fish oil (omega-3 fatty acids and specialized, small lipid pro-resolving epoxide derivatives), oxylipins, tart cherry, krill oil, astaxanthin, proteolytic enzymes, glucosamine sulfate, chondroitin sulfate, MSM (methylsulfonylmethane), SAMe (Sadenosylmethionine), ASU (avocado-soybean unsapponifiable fraction), cetyl myristoleate, Dolichos falcate, triterpenoids, acacia catechu, Andrographis paniculata, Scutalleria baicalensis, Agmatine sulfate, Stinging Nettle, Sea Buckthorn, Curcumin, Cissus Quadrilangularis, Boswellia Serrata, Wasabia japonica (wasabi extract for Tea Tree Oil), Emu Oil, Arnica, Mangifera indica L. (Anacardiaceae), Lagenaria breviflora, Zingiber officinale (ginger & gingerols/shogaols), hoodia gordonii, caffeine, yohimbine, methylsynephrine, synephrine, theobromine, flavenoids, tocopherols, theophylline, alphayohimbine, conjugated linoleic acid (CLA), octopamine, evodiamine, passion flower, red pepper, cayenne, raspberry ketone, guggul, green tea, guarana, kola nut, beta-Phenethylamines, Acacia rigidula, forskolin (Coleus forskohlli), theophylline, synephrine, yohimbine, rhodiola, ashwagandha, ginseng, Ginkgo biloba, siberian ginseng, astragalus, licorice, green tea, reishi, dehydroepiandrosterone (DHEA), pregnenolone, N-acetyl-, glucuronolactone, Acetyl-L-carnitine, 5-hydroxytryptophan, tryptophan, Phenethylamines, Sceletium tortuosum (and Mesembrine alkaloids), Dendrobium sp., Acacia rigidula, PQQ (Pyroloquinoline quinone), Ubiquinone(01), Nicotinamide riboside, picamilon, Huperzine A (Chinese clubmoss or Huperzia serrata, L-dopa, Mucuna pruriens, and forskolin (Coleus forskohlli), 2-(dimethylamino)ethanol (DMAE), DMAE bitartrate, Ornithine, Citrulline, Pyruvate, Eleutherococcus senticosus, D-Ribose, whey protein, Trimethylglycine, Arginine, HMB (β-hydroxy β-methylbutyrate), milk protein, Schisandra chinensis, Leucine, Betalains, Leucic Acid, L-Carnitine, Sodium Bicarbonate, Arachidonic acid, Beta-Alanine, Brassinosteroids, Hemp Protein, Alanylglutamine, Rhaponticum carthamoides, Casein, Ecdysteroids, Creatine, Branched-Chain Amino Acids, Beetroot, Coffee, Nitrate, Panax ginseng, Clenbuterol, Alpha-GPC, Valine, Colostrum, Trichopus zeylanicus, Ashwagandha, Terminalia arjuna, Eggs, Ursolic Acid, Isoleucine, medium-chain triglycerides, Glutamine, zinc, vitamin D, maca, Schizandra, nicotinamide mononucleotide (NMN), exogenous ketones, Ergothioneine, berberine, dihydroberberine and combinations thereof.
3. The composition of clause 1, further comprising a combination of paraxanthine and tyrosine congeners or combination of paraxanthine and tyrosine analogs.
4. The composition of clause 3, wherein the combination of paraxanthine congener or analog is selected from the group consisting of caffeine, 1-methylxanthine, combination of paraxanthine and 7-methylxanthine, paraxanthine, theobromine, theophylline, liberine, methylliberine, and combinations thereof.
5. The composition of clause 4, wherein the paraxanthine congener or analog is caffeine.
6. The composition of clause 5, wherein the effective dose of caffeine is lower than the effective dose of caffeine in a composition without combination of paraxanthine and tyrosine.
7. The composition of clause 3, wherein the tyrosine congener or analog is N-acetyl-L-tyrosine, glycyl-L-tyrosine, N-Acetyl-L-tyrosine ethyl ester or N-acetyl-L-tyrosine methyl ester.
8. The composition of clause 1, wherein tyrosine is present in polymeric form.
9. The composition of clause 8, wherein tyrosine is present as Dityrosine (Tyr-Tyr), Trityrosine (Tyr-Tyr-Tyr), Tetratyrosine (Tyr-Tyr-Tyr-Tyr) or a peptide containing the forgoing.
10. The composition of clause 8, wherein the tyrosine is present as Lysyltyrosine or Leucine-Tyrosine.
11. The composition of clause 8, wherein tyrosine is present in a dipeptide having the structure L-Tyr-X, wherein X is an amino acid.
12. The composition of any preceding clauses, wherein the paraxanthine and tyrosine are present at a ratio of from about 1:4 to about 1:30
13. The composition of clause 12, wherein the paraxanthine and tyrosine are present at a ratio of from about 1:4 to about 1:10.
14. A composition comprising paraxanthine and taurine.
15. The composition of clause 14, further comprising one or more active ingredient, selected from a group consisting of: gallic acid, (+)-catechin (C), (−)-epicatechin (EC), (+)-gallocatechin (GC), (−)-epigallocatechin (EGC), (−)-catechin gallate (CG), (−)-gallocatechin gallate (GCG), (−)-epicatechin gallate (ECG) and (−)-epigallocatechin gallate (EGCG), glycerides, propylene glycol, lauroyl macrogol, lauroyl macrogol derivatives, cocrystallization products of bioperine, piperine, black pepper, bergamottin, dihydroxybergamottin (CYP3A4), flavonoids (naringin, hesperidin, nobiletin, tangeretin, quercetin), pterostilbene, fisetin, phytosomes, salicin, fish oil (omega-3 fatty acids and specialized, small lipid pro-resolving epoxide derivatives), oxylipins, tart cherry, hill oil, astaxanthin, proteolytic enzymes, glucosamine sulfate, chondroitin sulfate, MSM (methylsulfonylmethane), SAMe (S adenosylmethionine), ASU (avocado-soybean unsapponifiable fraction), cetyl myristoleate, Dolichos falcate, triterpenoids, acacia catechu, Andrographis paniculata, Scutalleria baicalensis, Agmatine sulfate, Stinging Nettle, Sea Buckthorn, Curcumin, Cissus Quadrilangularis, Boswellia Serrata, Wasabia japonica (wasabi extract for Tea Tree Oil), Emu Oil, Arnica, Mangifera indica L. (Anacardiaceae), Lagenaria breviflora, Zingiber officinale (ginger & gingerols/shogaols), hoodia gordonii, caffeine, yohimbine, methylsynephrine, synephrine, theobromine, flavenoids, tocopherols, theophylline, alphayohimbine, conjugated linoleic acid (CLA), octopamine, evodiamine, passion flower, red pepper, cayenne, raspberry ketone, guggul, green tea, guarana, kola nut, beta-Phenethylamines, Acacia rigidula, forskolin (Coleus forskohlli), theophylline, synephrine, yohimbine, rhodiola, ashwagandha, ginseng, Ginkgo biloba, siberian ginseng, astragalus, licorice, green tea, reishi, dehydroepiandrosterone (DHEA), pregnenolone, N-acetyl-tyrosine, glucuronolactone, Acetyl-L-carnitine, 5-hydroxytryptophan, tryptophan, Phenethylamines, Sceletium tortuosum (and Mesembrine alkaloids), Dendrobium sp., Acacia rigidula, PQQ (Pyroloquinoline quinone), Ubiquinone(01), Nicotinamide riboside, picamilon, Huperzine A (Chinese clubmoss or Huperzia serrata, L-dopa, Mucuna pruriens, and forskolin (Coleus forskohlli), 2-(dimethylamino)ethanol (DMAE), DMAE bitartrate, Ornithine, Citrulline, Pyruvate, Eleutherococcus senticosus, D-Ribose, whey protein, Trimethylglycine, Arginine, HMB (β-hydroxy β-methylbutyrate), milk protein, Schisandra chinensis, Leucine, Betalains, Leucic Acid, L-Carnitine, Sodium Bicarbonate, Arachidonic acid, Beta-Alanine, Brassinosteroids, Hemp Protein, Alanylglutamine, Rhaponticum carthamoides, Casein, Ecdysteroids, Creatine, Branched-Chain Amino Acids, Beetroot, Coffee, Nitrate, Panax ginseng, Clenbuterol, Alpha-GPC, Valine, Colostrum, Trichopus zeylanicus, Ashwagandha, Terminalia arjuna, Eggs, Ursolic Acid, Isoleucine, medium-chain triglycerides, Glutamine, zinc, vitamin D, maca, Schizandra, nicotinamide mononucleotide (NMN), exogenous ketones, Ergothioneine, berberine, dihydroberberine and combinations thereof.
16. The composition of clauses 14-15 wherein paraxanthine and taurine a present at a ratio of about 1:5.
17. The composition of clauses 14-16, further comprising a combination of paraxanthine congeners and/or paraxanthine analogs.
18. The composition of clause 17, wherein the combination of paraxanthine congener or analog is selected from the group consisting of caffeine, 1-methylxanthine, combination of paraxanthine and 7-methylxanthine, paraxanthine, theobromine, theophylline, liberine, methylliberine, and combinations thereof.
19. The composition of clause 18, wherein the paraxanthine congener or analog is caffeine.
20. The composition of clause 19, wherein the effective dose of caffeine is lower than the effective dose of caffeine in a composition without combination of paraxanthine and taurine.
21. The composition of any preceding clause, wherein the composition is a powder.
22. The composition of any preceding clause, wherein the composition is a dietary supplement, and the supplement is in a solid oral dosage form.
23. The composition of any preceding clause, wherein the composition is formulated for topical administration.
24. The composition of any preceding clause, except clauses 5-6 and 19-20, wherein the composition is substantially free of caffeine.
25. A method for improving energy in subject, comprising: administering to the subject with the composition of clauses 1-24.
26. The method of clause 25, wherein upon administration of the composition, the subject experiences improvement of at least one of mood, energy, focus, concentration or sexual desire or a reduction of at least one of anxiety, fatigue, perception of effort or perception of pain.
27. The method of clause 26, wherein upon continued administration to the subject, the composition does not create dependence in the subject and/or withdrawal effect in the subject when continued use is ceased.
28. The method of clause 25, wherein the amount of paraxanthine provided is from about 50 mg to about 400 mg.
29. The method of clause 25, wherein the amount of tyrosine provided is from about 250 mg to about 13,500 mg.
30. The method of clause 25, wherein the amount of tyrosine provided is from about 100-150 mg/kg bodyweight of the subject.
31. The method of clause 25, wherein the subject experiences a decrease in fatigue of at least about 6 percent.
32. The method of clause 25, wherein the subject experiences an increase in energy of at least about 5 percent.
33. The method of clause 25, wherein the composition further comprises at least one ingredient selected from the group consisting of L-theanine, phosphatidylcholine, alpha-GPC (L-alpha glycerylphosphorylcholine), Citicoline (Cytidine diphosphate choline (CPD Choline)), Choline Bitartrate, Bacopa Monnieri, Phosphatidylserine, pilocarpine, and cevimeline Amburana cearensis, Lippia sidoides, Paullinia cupana, Plathymiscium floribundum, tetrahydrocurcumin, and Solanum asperum and/or combinations thereof, caffeine, theobromine, naringin, hesperidin, 2-(dimethylamino)ethanol (DMAE), DMAE bitartrate, magnolia bark, theanine, phosphatidylserine, ashwagandha, rhodiola, macuna, sceletium tortuosa, 5-HTP, tryptophan, saffron, Vitamin D, SAMe, lions mane and huperzine A.
34. A method of increasing athletic endurance in a subject comprising administering to the subject the composition of any of clauses 1-24.
35. The method of clause 34, wherein the composition is the composition of any of clauses 1-11, and wherein the administration of paraxanthine and tyrosine produce a synergistic increase in athletic endurance in the subject, relative to the administration of paraxanthine or tyrosine alone.
36. The method of clause 35, wherein the amount of tyrosine provided is from about 250 mg to about 13,500 mg.
37. The method of clause 35, wherein the amount of tyrosine provided is from about 100-150 mg/kg bodyweight of the subject.
38. The method of clause 34, wherein the composition is the composition of any of clauses 12-18, and wherein the administration of paraxanthine and taurine produce a synergistic increase in athletic endurance in the subject, relative to the administration of paraxanthine or taurine alone.
39. A method of treating a condition in a subject in need thereof, comprising administering to the subject the composition of any of clauses 1-24.
40. The method of clause 39, wherein the condition is selected from narcolepsy, epilepsy, attention deficit disorders, attention deficit hyperactivity syndrome (ADHD), cognitive deficit disorders, palsies, uncontrolled anger, migraine, substance abuse addictions, eating disorders, depression, anxiety disorders, traumatic head injury (TBI), concussion, Parkinson's disease, Alzheimer's, and dementia.
41. The method of clause 39, wherein the condition is a mood disorder.
42. The method of clause 41, wherein the mood disorder is depression.
43. The method of clause 42, wherein the subject has been diagnosed with depression or is at risk of depression.
44. The method of clause 40, wherein the condition is an anxiety disorder.
45. The method of clause 40, wherein the composition is administered in a therapeutically effective amount.
46. The method of clause 40, wherein the composition is administered in a prophylactically effective amount.
47. The method of clause 40, wherein the composition comprises paraxanthine at an amount from about 2 mg to about 800 mg.
48. The method of clause 39, wherein the composition further comprises at least one ingredient selected from the group consisting of L-theanine, phosphatidylcholine, alpha-GPC (L-alpha glycerylphosphorylcholine), Citicoline (Cytidine diphosphate choline (CPD Choline)), Choline Bitartrate, Bacopa Monnieri, Phosphatidylserine, pilocarpine, and cevimeline Amburana cearensis, Lippia sidoides, Paullinia cupana, Plathymiscium floribundum, tetrahydrocurcumin, and Solanum asperum and/or combinations thereof, caffeine, theobromine, naringin, hesperidin, 2-(dimethylamino) ethanol (DMAE), DMAE bitartrate, magnolia bark, theanine, phosphatidylserine, ashwagandha, rhodiola, n-acetyl-tyrsoine, macuna, sceletium tortuosa, 5-HTP, tryptophan, saffron, Vitamin D, SAMe, lions mane and/or huperzine A.
49. A method of enhancing attention in a subject in need thereof comprising administering the composition of any of clauses 1-24.
50. A method of improving working memory in a subject in need thereof comprising administering a composition to the subject comprising the composition of any of clauses 1-24.
51. A method of improving cognitive performance in a subject comprising administering the composition of any of clause 1-24.
52. The method of clause 51, wherein improved cognitive function is measured by an increase in one or more of: attention, information acquisition, information processing, working memory, short-term memory, long-term memory, anterograde memory, retrograde memory, memory retrieval, discrimination learning, decision-making, inhibitory response control, attentional set-shifting, delayed reinforcement learning, reversal learning, the temporal integration of voluntary behavior, speed of processing, reasoning, problem solving and/or social cognition.
53. A method for increasing muscle function in a subject, comprising:
administering to the subject the composition of any of clauses 1-24.
54. The method of clause 53, wherein the composition further comprises one or more compounds selected from the list consisting of: isoleucine, leucine, and valine, histidine, isoleucine, leucine, lysine, methionine, phenylalanine, threonine, tryptophan, valine, creatine, arginine, cysteine, glutamine, glycine, proline, carnitine, beta-alanine, and beta-hydroxy beta-methylbutyrate.
55. A nutritional supplement for improving muscle strength, muscle size, and/or muscle function comprising the composition of any of clauses 1-24.
56. The nutritional supplement of clause 55, wherein the nutritional supplement is powder or a capsule.
57. The nutritional supplement of clause 55, wherein the nutritional supplement is a functional food.
58. The nutritional supplement of clause 57, wherein the functional food is a beverage, nutrition bar, yoghurt, or cereal.
59. The nutritional supplement clause 55, further comprises one or more compounds selected from the list consisting of: isoleucine, leucine, and valine, histidine, isoleucine, leucine, lysine, methionine, phenylalanine, threonine, tryptophan, valine, creatine, arginine, cysteine, glutamine, glycine, proline, carnitine, beta-alanine, beta-hydroxy beta-methylbutyrate, L-arginine, Omega-3 fatty acids, Vitamin D, whey protein, and other protein extracts from animal, plant or fermentation sources.
60. A method of increasing muscle size in a subject comprising administering to the subject in need thereof with an effective amount of the composition of any of clauses 1-24.
61. The method of clause 60, wherein the composition is the composition of any of clauses 1-11, and wherein the administration of paraxanthine and tyrosine produce a synergistic increase in muscle size in the subject, relative to the administration of paraxanthine or tyrosine alone.
62. The method of clause 61, wherein the amount of tyrosine provided is from about 250 mg to about 13,500 mg.
63. The method of clause 61, wherein the amount of tyrosine provided is from about 100-150 mg/kg bodyweight of the subject.
64. The method of clause 60, wherein the composition is the composition of any of clauses 12-18, and wherein the administration of paraxanthine and taurine produce a synergistic increase in muscle size in the subject, relative to the administration of paraxanthine or taurine alone.
65. A method for increasing energy in a subject comprising administering to the subject in need thereof with an effective amount of the composition of any of clauses 1-24.
66. The method of clause 65, wherein the amount of paraxanthine administered is from about 25 mg to about 800 mg.
67. The method of clause 65, wherein the subject experiences and increase in perception of energy of at least about 5%.
68. The method of clause 65, wherein the subject experiences a decrease of at least one of anxiety, fatigue, perception of effort, and/or perception of pain.
69. The method of clause 65, wherein the composition further comprises paraxanthine in an amount from about 2 mg to about 800 mg.
70. The method of clause 69, wherein the composition is the composition of any of clauses 1-11, and wherein the administration of paraxanthine and tyrosine produce a synergistic increase in perception of energy in the subject, relative to the administration of a comparable dose of paraxanthine or tyrosine alone.
71. The method of clause 70, wherein the amount of tyrosine provided is from about 250 mg to
72. The method of clause 70, wherein the amount of tyrosine provided is from about 100-150 mg/kg bodyweight of the subject.
73. The method of clause 69, wherein the composition is the composition of any of clauses 12-18, and wherein the administration of paraxanthine and taurine produce a synergistic increase in perception of energy in the subject, relative to the administration of a comparable dose of paraxanthine or taurine alone.
74. The method of clause 65, wherein the composition further comprises at least one ingredient selected from the group consisting of L-theanine, phosphatidylcholine, alpha-GPC (L-alpha glycerylphosphorylcholine), Citicoline (Cytidine diphosphate choline (CPD Choline)), Choline Bitartrate, Bacopa Monnieri, Phosphatidylserine, pilocarpine, and cevimeline Amburana cearensis, Lippia sidoides, Paullinia cupana, Plathymiscium floribundum, tetrahydrocurcumin, and Solanum asperum and/or combinations thereof, caffeine, theobromine, naringin, hesperidin, 2-(dimethylamino)ethanol (DMAE), DMAE bitartrate, magnolia bark, theanine, phosphatidylserine, ashwagandha, rhodiola, n-acetyl-tyrsoine, macuna, sceletium tortuosa, 5-HTP, tryptophan, saffron, Vitamin D, SAMe, lions mane and huperzine A.
75. The method of clause 65, wherein the composition is substantially free of caffeine.
76. A method for improving athletic performance in a subject in comprising administering to the subject a composition comprising an effective amount the composition of any of clauses 1-24.
77. The method of clause 75, wherein the amount of paraxanthine administered is from about 50 mg to about 400 mg.
78. The method of clause 76, wherein athletic performance is increased by at least about 10%.
79. The method of clause 76, wherein the subject experiences and increase in endurance.
80. The method of clause 76, wherein the composition is the composition of any of clauses 1-11, and wherein administration of the composition to a subject produces a synergistic increase in athletic performance to the administration of a comparable dose of paraxanthine or tyrosine alone.
81. The method of clause 76, wherein the composition is the composition of any of clauses 12-18, and wherein administration of the composition to a subject produces a synergistic increase in athletic performance to the administration of a comparable dose of paraxanthine or taurine alone.
82. The method of clause 76, wherein the composition further comprises at least one agent selected from the group consisting of L-theanine, phosphatidylcholine, alpha-GPC (L-alpha glycerylphosphorylcholine), Citicoline (Cytidine diphosphate choline (CPD Choline)), Choline Bitartrate, Bacopa Monnieri, Phosphatidylserine, pilocarpine, and cevimeline Amburana cearensis, Lippia sidoides, Paullinia cupana, Plathymiscium floribundum, tetrahydrocurcumin, and Solanum asperum and/or combinations thereof, caffeine, theobromine, naringin, hesperidin, 2-(dimethylamino)ethanol (DMAE), DMAE bitartrate, huperzine A, theacrine, methylliberine, B12, sulbutiamine, magnolia bark, ketones, MCTs, omega 3's, lutein, zeaxanthin, and n-acetyl-tyrosine, acetyl-1-carnitine and/or combinations thereof.

### EXAMPLES

The following examples are put forth so as to provide those of ordinary skill in the art with a complete disclosure and description of certain examples of how the compounds, compositions, articles, devices and/or methods claimed herein are made and evaluated, and are intended to be purely exemplary of the invention and are not intended to limit the scope of what the inventors regard as their invention. However, those of skill in the art should, in light of the present disclosure, appreciate that many changes can be made in the specific embodiments which are disclosed and still obtain a like or similar result without departing from the spirit and scope of the invention.

### Example 1

### Paraxanthine Plus Tyrosine

### Cognition, Memory, Learning

### 1.1. Methods

Behavioral studies were conducted in mice to examine the learning and memory ability by using Cook's pole test.

Thirty-two 8-week-old male Swiss Albino mice were housed in an animal room at a constant temperature (22±3° C.) and humidity (30%-70%) under a 12:12 h light-dark cycle with standard laboratory diet (Purina 5L79, Rat and Mouse 18% protein; PMI Nutrition International, Brentwood, Mo., USA). Distilled water was provided ad libitum. All animal experiments were reviewed and approved by the Institutional Animal Ethical Committee (IAEC) of Radiant Research Services Pvt. Ltd (Bangalore, India). All research was conducted in accordance with the guidelines of the committee for the purpose of control and supervision of experiments on animals.

After one week of acclimation, the animals were randomly divided by body weight into four groups (n=8 per group in each test) for oral treatment once a day, at approximately same time each day (±1 hour), for 7 consecutive days: (1) vehicle control or (2) paraxanthine or (3) tyrosine or (4) tyrosine plus paraxanthine. The dose administered to the mice was calculated using US Food and Drug Administration for human equivalence doses (HED), assuming a human weight of 60 kg. The following HED was used in this study: 100 mg paraxanthine (ENFINITY®, Ingenious Ingredients, L.P Lewisville, Tex., USA; mouse dose: 20.5 mg/kg bw/day) or 500 mg tyrosine (mouse dose: 102.75 mg/kg bw/day), or 500 mg tyrosine (mouse dose: 102.75 mg/kg bw/day) plus 100 mg paraxanthine (ENFINITY®, Ingenious Ingredients, L.P Lewisville, Tex., USA; mouse dose: 20.5 mg/kg bw/day). 0.5% Carboxy Methyl Cellulose sodium was used as vehicle and the test item formulations were prepared daily. Dosing was conducted via oral gavage using disposable polypropylene syringes with sterilized stainless steel gavage tubes. Food intake was monitored daily while water intake was ad libitum.

### 1.2. Cook's Pole Climbing Test

Mice were trained in such a way that the animal had to climb the pole (shock free zone) within 30 seconds to avoid a shock. The shock was preceded by a buzzer that lasted for 15 seconds. The animals were trained to climb the pole at the sound of the buzzer (conditioned avoidance response). At particular intervals, 20 trials were given for each animal and average of the shock avoidance and mistakes were recorded. Trained animals were assayed by conditioned avoidance responses.

### 1.3. Induction of Amnesia

Amnesia was induced by using the scopolamine injection. Scopolamine is an anti-cholinergic and is an attractive amnesic agent for discerning the action of candidate anti amnesic drugs. Scopolamine is a non-selective post synaptic muscarinic receptor blocker and can cause cognitive impairments in rodents and humans via decreasing the effectiveness of ACH in the CNS in animals and humans. Scopolamine can induce the significant deficits in cognitive performance on behavioral tests which makes it a valid pharmacological model for inducing cognitive deficits. In this study to evaluate the cognitive effect, the mice were injected with scopolamine intraperitoneally to induce memory deficits.

### 2. Results

The mice treated with paraxanthine showed reversal of amnesia induced by scopolamine and improved memory and learning. The combination of paraxanthine and tyrosine showed synergistic effects over paraxanthine or tyrosine alone.

### 2.1. Effect of Supplementation on Cook's Pole Climbing Test

Escape latency in the paraxanthine group (16.38±2.33 seconds) was 46.3% faster than control (6.38±1.41 seconds). Escape latency in the tyrosine group (8.25±1.04 seconds) was 30.6% faster than control, and 29.3% slower than paraxanthine. Escape latency in the paraxanthine plus tyrosine group (5.88±0.83 seconds) was 50.5% faster than control, 7.8% faster than paraxanthine alone, and 28.7% faster than tyrosine alone.

### Example 2

### Paraxanthine Plus Tyrosine

### Sports & Active Nutrition, Athletic Performance, Strength, Energy, Mood

### 1.1. Methods

Thirty-two 8-week-old male Swiss Albino mice were housed in an animal room at a constant temperature (22±3° C.) and humidity (30%-70%) under a 12:12 h light-dark cycle with standard laboratory diet (Purina 5L79, Rat and Mouse 18% protein; PMI Nutrition International, Brentwood, Mo., USA). Distilled water was provided ad libitum. All animal experiments were reviewed and approved by the Institutional Animal Ethical Committee (IAEC) of Radiant Research Services Pvt. Ltd (Bangalore, India). All research was conducted in accordance with the guidelines of the committee for the purpose of control and supervision of experiments on animals.

After one week of acclimation, the animals were randomly divided by body weight into four groups (n=8 per group in each test) for oral treatment once a day, at approximately same time each day (±1 hour), for 28 consecutive days: (1) vehicle control or (2) paraxanthine or (3) tyrosine or (4) tyrosine plus paraxanthine. The dose administered to the mice was calculated using US Food and Drug Administration for human equivalence doses (HED), assuming a human weight of 60 kg. The following HED was used in this study: 100 mg paraxanthine (ENFINITY®, Ingenious Ingredients, L.P Lewisville, Tex., USA; mouse dose: 20.5 mg/kg bw/day) or 500 mg tyrosine (mouse dose: 102.75 mg/kg bw/day), or 500 mg tyrosine (mouse dose: 102.75 mg/kg bw/day) plus 100 mg paraxanthine (ENFINITY®, Ingenious Ingredients, L.P Lewisville, Tex., USA; mouse dose: 20.5 mg/kg bw/day). 0.5% Carboxy Methyl Cellulose sodium was used as vehicle and the test item formulations were prepared daily. Dosing was conducted via oral gavage using disposable polypropylene syringes with sterilized stainless steel gavage tubes. Food intake was monitored daily while water intake was ad libitum.

### 1.2. Forelimb Grip Strength Test

The forelimb grip strength was measured on day 0 and day 28 by using a stainless-steel grill to assess muscle strength (Orchid Scientific & Innovative India Pvt Ltd, India). Grip strength was measured one hour after treatment. Briefly, each mouse was first placed in the testing room for ten minutes to acclimate. Each mouse was then placed over the top of the grid of a grip-strength meter to allow the mouse to grasp the grid with all four paws. The mouse was held by the base of the tail without pressing down upon the grid. The animal was then gently pulled backwards away from the grid by the tail pulling along the axis of the grip strength measurement. The speed was slow enough to let the mouse to develop a resistance against the pulling force and the score that is displayed (gf) on the screen of the grip strength measurement was recorded once the mouse released the grid. Each animal performed three independent trials and the mean of the three trials was calculated and recorded.

### 1.3. Exercise Training

During treatment period, training was accomplished on a swimming chamber at a moderate room temperature for 15 mins. The animals were adapted to this procedure daily 1 hr after dosing for 5 days in a week during the treatment period. On 28th day of respective treatment of all the animals were subjected to force swim test. Animals were forced to swim individually for 30 min in a glass jar of height 20 cm, diameter 10 cm, and filled with fresh water to a depth of 15 cm at room temperature. The parameters measured were total time spent in active swimming (the total duration during which the animal swims throughout the experimental period).

### 2. Results

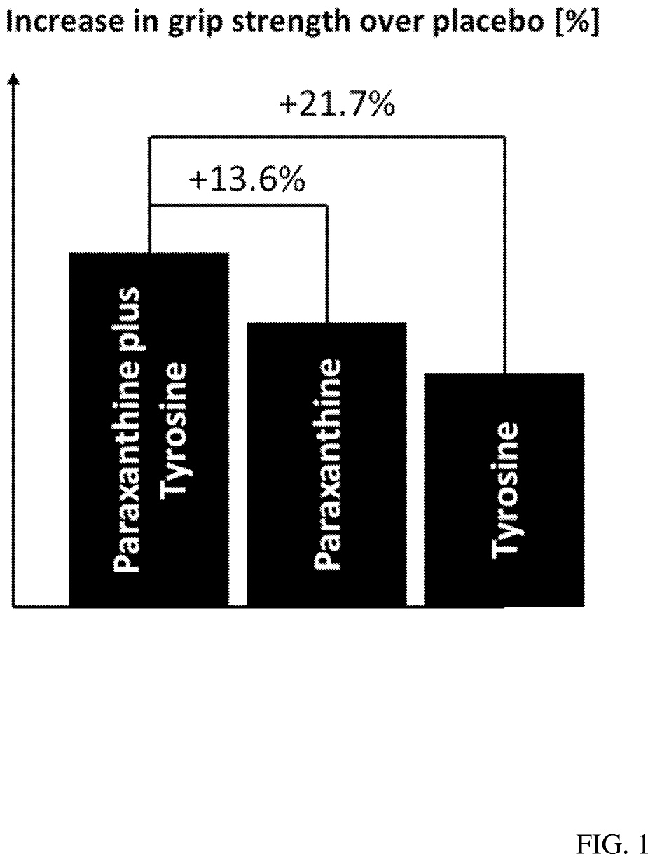

As shown in FIG. 1, mice treated with paraxanthine showed improved strength over control. Tyrosine improved strength at a lesser degree, however, the combination of paraxanthine and tyrosine showed synergistic effects over paraxanthine or tyrosine alone.

### 2.1. Effect of Supplementation on Forelimb Grip Strength

### 2.2. Effect of Supplementation on Energy and Mood

The mice treated with paraxanthine showed improved energy and mood. The combination of paraxanthine and tyrosine showed synergistic benefits over paraxanthine and tyrosine alone.

The duration of mobility/active swimming was synergistically greater in the combination of paraxanthine and tyrosine group (9.38±1.30 minutes) compared to control (8.75±1.39 minutes), +7.2%, tyrosine (8.88±1.46 minutes) and paraxanthine (8.63±1.92 minutes).

### Example 3

### Paraxanthine Plus Taurine

### Energy, Mood

### 1.1. Methods

Twenty-four 8-week-old male Swiss Albino mice were housed in an animal room at a constant temperature (22±3° C.) and humidity (30%-70%) under a 12:12 h light-dark cycle with standard laboratory diet (Purina 5L79, Rat and Mouse 18% protein; PMI Nutrition International, Brentwood, Mo., USA). Distilled water was provided ad libitum. All animal experiments were reviewed and approved by the Institutional Animal Ethical Committee (IAEC) of Radiant Research Services Pvt. Ltd (Bangalore, India). All research was conducted in accordance with the guidelines of the committee for the purpose of control and supervision of experiments on animals.

After one week of acclimation, the animals were randomly divided by body weight into three groups (n=8 per group in each test) for oral treatment once a day, at approximately same time each day (±1 hour), for 28 consecutive days: (1) paraxanthine or (2) taurine or (3) taurine plus paraxanthine. The dose administered to the mice was calculated using US Food and Drug Administration for human equivalence doses (HED), assuming a human weight of 60 kg. The following HED was used in this study: 100 mg paraxanthine (ENFINITY®, Ingenious Ingredients, L.P Lewisville, Tex., USA; mouse dose: 20.5 mg/kg bw/day) or 500 mg taurine (mouse dose: 102.75 mg/kg bw/day), or 500 mg taurine (mouse dose: 102.75 mg/kg bw/day) plus 100 mg paraxanthine (ENFINITY®, Ingenious Ingredients, L.P Lewisville, Tex., USA; mouse dose: 20.5 mg/kg bw/day). 0.5% Carboxy Methyl Cellulose sodium was used as vehicle and the test item formulations were prepared daily. Dosing was conducted via oral gavage using disposable polypropylene syringes with sterilized stainless steel gavage tubes. Food intake was monitored daily while water intake was ad libitum.

### 1.2. Exercise Training

During treatment period, training was accomplished on a swimming chamber at a moderate room temperature for 15 mins. The animals were adapted to this procedure daily 1 hr after dosing for 5 days in a week during the treatment period. On 28th day of respective treatment of all the animals were subjected to force swim test. Animals were forced to swim individually for 30 min in a glass jar of height 20 cm, diameter 10 cm, and filled with fresh water to a depth of 15 cm at room temperature. The parameter measured was total time spent in active swimming (the total duration during which the animal swims throughout the experimental period) and duration of immobility (the total time during which the animal is immobile).

### 2. Results

The mice treated the combination of paraxanthine and taurine showed synergistic effects over paraxanthine or taurine alone.

### 2.1. Effect of Supplementation on Energy and Mood

The duration of mobility/active swimming was synergistically greater in the combination of paraxanthine and taurine group (8.75±1.04 minutes) compared to taurine (8.50±2.20 minutes) and paraxanthine (8.63±1.92 minutes).

The duration of immobility was synergistically lower in the combination of paraxanthine and taurine group (21.25±1.04 minutes) compared to taurine (21.50±2.20 minutes) and paraxanthine (21.38±1.92 minutes).

While multiple embodiments are disclosed, still other embodiments of the disclosure will become apparent to those skilled in the art from the following detailed description, which shows and describes illustrative embodiments of the disclosed compositions, systems and methods. As will be realized, the disclosed compositions, systems and methods are capable of modifications in various obvious aspects, all without departing from the spirit and scope of the disclosure. Accordingly, the drawings and detailed description are to be regarded as illustrative in nature and not restrictive.

What is claimed is:

1. A dietary supplement comprising paraxanthine and tyrosine, wherein the paraxanthine and tyrosine are present in a ratio from about 1:4 to about 1:30.

2. The dietary supplement of claim 1, further comprising a further active ingredient, selected from a group consisting of: gallic acid, (+)-catechin (C), (−)-epicatechin (EC), (+)-gallocatechin (GC), (−)-epigallocatechin (EGC), (−)-catechin gallate (CG), (−)-gallocatechin gallate (GCG), (−)-epicatechin gallate (ECG) and (−)-epigallocatechin gallate (EGCG), glycerides, propylene glycol, lauroyl macrogol, lauroyl macrogol derivatives, cocrystallization products of bioperine, piperine, black pepper, bergamottin, dihydroxybergamottin (CYP3A4), flavonoids (naringin, hesperidin, nobiletin, tangeretin, quercetin), pterostilbene, fisetin, phytosomes, salicin, fish oil (omega-3 fatty acids and specialized, small lipid pro-resolving epoxide derivatives), oxylipins, tart cherry, hill oil, astaxanthin, proteolytic enzymes, glucosamine sulfate, chondroitin sulfate, MSM (methylsulfonylmethane), SAMe (Sadenosylmethionine), ASU (avocado-soybean unsapponifiable fraction), cetyl myristoleate, Dolichos falcate, triterpenoids, acacia catechu, Andrographis paniculata, Scutalleria baicalensis, Agmatine sulfate, Stinging Nettle, Sea Buckthorn, Curcumin, Cissus Quadrilangularis, Boswellia Serrata, Wasabia japonica (wasabi extract for Tea Tree Oil), Emu Oil, Arnica, Mangifera indica L. (Anacardiaceae), Lagenaria breviflora, Zingiber officinale (ginger & gingerols/shogaols), hoodia gordonii, caffeine, yohimbine, methylsynephrine, synephrine, theobromine, flavenoids, tocopherols, theophylline, alphayohimbine, conjugated linoleic acid (CLA), octopamine, evodiamine, passion flower, red pepper, cayenne, raspberry ketone, guggul, green tea, guarana, kola nut, beta-Phenethylamines, Acacia rigidula, forskolin (Coleus forskohlli), theophylline, synephrine, yohimbine, rhodiola, ashwagandha, ginseng, Ginkgo biloba, siberian ginseng, astragalus, licorice, green tea, reishi, dehydroepiandrosterone (DHEA), pregnenolone, N-acetyl-tyrosine, glucuronolactone, Acetyl-L-carnitine, 5-hydroxytryptophan, tryptophan, Phenethylamines, Sceletium tortuosum (and Mesembrine alkaloids), Dendrobium sp., Acacia rigidula, PQQ (Pyroloquinoline quinone), Ubiquinone(01), Nicotinamide riboside, picamilon, Huperzine A (Chinese clubmoss or Huperzia serrata, L-dopa, Mucuna pruriens, and forskolin (Coleus forskohlli), 2-(dimethylamino)ethanol (DMAE), DMAE bitartrate, Ornithine, Citrulline, Pyruvate, Eleutherococcus senticosus, D-Ribose, whey protein, Trimethylglycine, Arginine, HMB (β-hydroxy β-methylbutyrate), milk protein, Schisandra chinensis, Leucine, Betalains, Leucic Acid, L-Carnitine, Sodium Bicarbonate, Arachidonic acid, Beta-Alanine, Brassinosteroids, Hemp Protein, Alanylglutamine, Rhaponticum carthamoides, Casein, Ecdysteroids, Creatine, Branched-Chain Amino Acids, Beetroot, Coffee, Nitrate, Panax ginseng, Clenbuterol, Alpha-GPC, Valine, Colostrum, Trichopus zeylanicus, Ashwagandha, Terminalia arjuna, Eggs, Ursolic Acid, Isoleucine, medium-chain triglycerides, Glutamine, zinc, vitamin D, maca, Schizandra, nicotinamide mononucleotide (NMN), exogenous ketones, Ergothioneine, berberine, dihydroberberine and combinations thereof.

3. The dietary supplement of claim 1, further comprising a combination of paraxanthine and tyrosine congeners or combination of paraxanthine and tyrosine analogs.

4. The dietary supplement of claim 3, wherein the tyrosine congener or analog is N-acetyl-L-tyrosine, glycyl-L-tyrosine, N-Acetyl-L-tyrosine ethyl ester or N-acetyl-L-tyrosine methyl ester.

5. The dietary supplement of claim 1, wherein tyrosine is present in polymeric form and wherein the polymeric form is Dityrosine (Tyr-Tyr), Trityrosine (Tyr-Tyr-Tyr), Tetratyrosine (Tyr-Tyr-Tyr-Tyr) or a peptide containing the forgoing.

7. The dietary supplement of claim 5, wherein the tyrosine is present as Lysyltyrosine or Leucine-Tyrosine.

8. The dietary supplement of claim 5, wherein tyrosine is present in a dipeptide having the structure L-Tyr-X, wherein X is an amino acid.

9. A method for athletic performance or energy in subject, comprising:
administering to the subject a composition comprising an effective amount of paraxanthine and tyrosine.

10. The method of claim 9, wherein the administration of paraxanthine and taurine produce a synergistic increase in athletic performance or energy in the subject, relative to the administration of paraxanthine or taurine alone.

11. The method of claim 9, wherein paraxanthine is provided in an amount of from about 25 mg to about 400 mg and wherein tyrosine is provided in an amount 100-150 mg/kg bodyweight of the subject.

12. The method of claim 11, wherein the subject experiences increased endurance or increased strength.

13. The method of claim 9, wherein ratio of the amount of paraxanthine and tyrosine administered to the subject is from about 1:10 to about 1:30.

14. The method of claim 9, wherein the composition is substantially free of caffeine.

15. A method of improving cognitive function in a subject comprising administering to the subject a composition comprising an effective amount of paraxanthine and tyrosine.

16. The method of claim 15, wherein improved cognitive function is measured by an increase in one or more of: attention, information acquisition, information processing, working memory, short-term memory, long-term memory, anterograde memory, retrograde memory, memory retrieval, discrimination learning, decision-making, inhibitory response control, attentional set-shifting, delayed reinforcement learning, reversal learning, the temporal integration of voluntary behavior, speed of processing, reasoning, problem solving and/or social cognition.

17. The method of claim 15, wherein ratio of the amount of paraxanthine and tyrosine administered to the subject is from about 1:10 to about 1:30.

18. The method of claim 15, wherein administration of the composition to the subject enhances mood in the subject.

19. The method of claim 15, wherein the administration of paraxanthine and tyrosine produce a synergistic enhancement in cognitive function in the subject, relative to the administration of paraxanthine or tyrosine alone.

20. A method of enhancing energy or mood in a subject comprising administering to the subject a composition comprising an effective amount of paraxanthine and taurine, wherein the amount for paraxanthine administered to the subject is from about 25 mg to about 800 mg and wherein the amount for taurine administered to the subject is from about 100 mg to about 6000 mg and wherein the administration of paraxanthine and taurine produce a synergistic enhancement in energy and/or mood in the subject, relative to the administration of paraxanthine or taurine alone.
